(19) United States
(12) Patent Application Publication
(10) Pub. No.: US 2020/0248119 A1
(43) Pub. Date: Aug. 6, 2020

(54) MICROBIOLOGICAL CULTURE DEVICE COMPRISING A SHEET OF DEHYDRATED POLYSACCHARIDE HYDROGEL

(71) Applicant: BIOMÉRIEUX, Marcy L'Etoile (FR)

(72) Inventors: Marie-Pierre MONTET, Lyon (FR); Christine ROZAND, Saint-Genis-les-Ollières (FR); Jean-Pierre FLANDROIS, Lyon (FR)

(73) Assignee: BIOMÉRIEUX, Marcy L'Etoile (FR)

(21) Appl. No.: 16/603,484

(22) PCT Filed: Apr. 24, 2018
(86) PCT No.: PCT/FR2018/051028

§ 371 Date: Oct. 7, 2019

(30) Foreign Application Priority Data
Apr. 26, 2017 (FR) 1753616

(51) Int. Cl. C12M 1/12; C08B 37/00

(52) U.S. Cl. CPC C12M 25/02; C08B 37/0033; C08B 37/006

(57) ABSTRACT

A microbiological culture device including: a part made of absorbent material having an at least substantially planar upper face and incorporating into its thickness a dehydrated culture medium composition, resting on the part made of absorbent material, a sheet of dehydrated polysaccharide hydrogel which can be rehydrated at temperatures of between 5° C. and 40° C.; said sheet of dehydrated hydrogel being affixed directly on the upper face of the part made of absorbent material, or indirectly, through a permeable membrane insert.

The present invention belongs to the field of microbiology and of the culture of microorganisms on solid and semi-solid media. It relates more specifically to alternative solutions proposed to traditional agar-based culture media, intended especially for the culture, detection and/or identification of microorganisms present in a sample to be analyzed.

In the field of clinical or veterinary diagnostics, and also that of industrial microbiological testing (in particular in the food-processing, pharmaceutical and cosmetics industries), solid and semi-solid culture supports—more commonly referred to as agar-based (culture) media or nutrient agar—constitute indispensable tools for detecting and studying potentially pathogenic and/or infectious microorganisms. Solidified culture media appeared at the end of the 19th century with the use of agar agar as gelling agent and they rapidly and profoundly revolutionized microbiological practices. Since then, microorganisms to detect, identify and/or study have been found, observed and handled on the macroscopic scale in the form of colonies, that is to say clusters of cells visible to the naked eye and growing on the surface of these solid culture media. In so doing, new analytical and experimental perspectives have come to light, and improvements and simplifications of existing techniques have been made possible.

In this regard, mention will most particularly be made of techniques for isolating microorganisms on agar-based medium, which still remain very widely used to this day in numerous methods for microbiological analyses, given their highly simple implementation. These are essentially techniques for isolation, by exhaustion or by the quadrant technique.

These techniques for isolation on agar-based medium consist in spreading, over the surface of the solid culture medium, the cells from a deposit of biological sample to be analyzed. This spreading is carried out for example using mechanical tools/means that are slid over the surface of the culture medium following a particular pattern. At the end of this spreading process, the cells that have reached the end of the streaking pattern are individualized, separated from one another. Each of these individualized cells then grows to eventually form a pure culture colony, that is to say a cluster of cells containing microorganisms that are all genetically identical. Each colony can then be subject to morphological analysis (examination of the shape of the colony, the height thereof, the regularity of the edges, etc.) directly on this same culture medium or on other media. These cells can then be collected for the purposes of preservation and/or with a view to supplemental subsequent analyses.

Aside from offering microorganisms a suitable support for their growth and development, gelled/solid culture media, conventionally agar-based, have the advantage of being able to have hardness levels and surface states making them perfectly suitable for operations of isolation and streaking of cells, and in particular for the forces of pressure and friction exerted by the tools and instruments developed for this purpose (cf. for example EP 0 242 114, WO 2005/071055).

However, agar-based culture media have some major disadvantages. To this end, mention will be made of a preparation which, when it is hand-made, requires time (at least one hour) and numerous operations (weighing ingredients, dissolutions, autoclaving, pouring, spreading out in Petri dishes). In addition, as is the case for liquid culture media, these solid or semi-solid media are extremely sensitive to any form of biological contamination. Finally, due to the great difficulty of ensuring the sterility thereof, “hand-made” agar-based media must be prepared extemporaneously or virtually extemporaneously. Industrially produced agar-based media also exist. These ready-to-use agar-based media have short expiration durations, which barely exceed a few months. This limited expiration duration may especially be explained in part by the presence of water in the media. Moreover, in order to guarantee optimal sterility and an acceptable level of performance thereof, they are subjected to excessive measures in terms of packaging (UV sterilizing treatment, double packages, etc.), of transport and of preservation (storage between 2° C. and 8° C., away from light). These numerous constraints have an impact on the sales and usage costs of industrial agar-based media.

In order to overcome the abovementioned disadvantages, alternatives to agar-based culture media have been proposed. These are in particular industrially produced, ready-to-use microbiological culture devices which have the particular feature of integrating nutrient compositions, which are optionally selective and/or differential, and which are dehydrated and can be activated simply by (re)hydration. As a result, aside from relatively non-restrictive preservation/storage requirements (away from moisture and high temperatures), the expiration duration thereof may reach several years.

In this regard, 3M (United States) proposes a range of industrial microbiological culture devices named Petrifilm™. These devices, especially described in EP 0 070 310, EP 0 620 844 or else EP 0 832 180, are formed of two watertight films affixed to one another. At the interface, the inner face of each of the two films is covered with an adhesive composition making it possible to adhere thereto a thin layer of powder(s) water-soluble under cold conditions.

For some of the Petrifilm™ devices, one of the inner faces of the films is covered with a first powder, which corresponds to a dehydrated and micronized culture medium composition. The other inner face is covered with a second powder which corresponds to a micronized gelling agent (such as guar gum and/or xanthan gum).

For other Petrifilm™ devices, the two inner faces are covered with the same powder, formed from a mixture of a dehydrated and micronized culture medium composition and an also micronized gelling agent.

These Petrifilm™ devices are intended for the detection and/or counting of microorganisms present in a sample to be analyzed. For this purpose, the culture device is opened by raising the upper transparent film. A volume of sample to be analyzed (liquid or previously liquidized) with a high degree of fluidity, optionally diluted and fluidized beforehand, is deposited in the center of the lower film. The upper film is repositioned on the lower film. On contact with the water present in the sample to be analyzed, the gelling agent forms a hydrogel with a high water content, having a gelatinous and viscous appearance, which sticks to the cells present in the sample to be analyzed. This same water present in the sample to be analyzed also makes it possible to dissolve and activate the culture medium. By gently compressing the sample between the two films, the sample is spread over a larger culture surface area. The microbiological culture device is finally incubated at the prescribed temperature and for the prescribed duration before reading the results.

Due to the design thereof, while the Petrifilm™ devices do indeed enable good fixation of the cells, the hydrogel formed creates a highly gelatinous and viscous surface which does not lend itself to operations of isolation and/or streaking of cells such as could be carried out on an agar-based culture medium, and moreover is not compatible with the tools and instruments developed for this purpose (cf. for example EP 0 242 114, WO 2005/071055).

The culture devices Compact Dry™, developed by NISSUI PHARMACEUTICAL (Japan) are also known, the microbiological culture support of which is formed from a sheet of absorbent fibrous material, incorporating, within the substance thereof, a dehydrated nutrient medium, which is optionally also selective and/or differential (cf. for example EP 1 179 586).

To this end, an alcohol-based suspension is prepared by mixing, in ethanol, a culture medium composition, an adhesive that is soluble both in water and in ethanol (for example poly(ethylene oxide) and hydroxypropyl cellulose), and a gelling agent that is soluble in water but insoluble in ethanol (for example locust bean gum, guar gum, carrageenan, hydroxyethyl cellulose). This alcohol-based suspension is used to impregnate the sheet of absorbent fibrous material. After drying, said sheet forms a ready-to-use dehydrated culture support with a long shelf life, that can be activated simply by rehydration.

These Compact Dry devices are intended for the detection and/or counting of microorganisms present in a sample to be analyzed. To this end, a volume of sample to be analyzed (liquid or previously liquidized) with a high degree of fluidity, optionally diluted beforehand, is inoculated using a pipette on the culture support, covering as much surface area as possible. The culture device is then incubated at the prescribed temperature and for the prescribed duration before reading the results.

This type of culture support does not lend itself to isolation techniques carried out by streaking the samples to be analyzed. This is because, due to the fibrous nature thereof, these culture supports have an irregular surface having numerous rough areas which hinder the continuous and regular sliding of the tools and instruments conventionally used to streak and spread the cells over the surface of a conventional agar-based medium. In addition, the cells tend to grow deep within the thickness of the porous support, which may make it difficult to visualize/identify the colonies formed.

In WO 2015/104501, the applicant also proposes ready-to-use microbiological culture devices intended for the detection, identification and/or counting of microorganisms. These devices are also based on a culture support made of fibrous absorbent material, incorporating in the thickness thereof a culture medium composition. The dehydrated culture medium composition is incorporated into the thickness of the culture support by a dry impregnation technique. As for the Compact Dry™ devices, due to the fibrous nature thereof, these culture supports have an irregular surface with numerous rough areas; these devices are therefore not compatible with the techniques and tools hitherto developed for the isolation and streaking of cells over the surface of a conventional agar-based medium.

In addition, as for the Compact Dry™ devices, the cells grow deep in the thickness of the porous support.

In order to overcome these drawbacks, in WO 2014/013089 and WO 2015/107228 the applicant proposes covering this fibrous surface with a surface layer which is able to perfect the surface state.

WO 2014/013089 thus proposes using a (micro)filtration membrane with sufficiently narrow pores to prevent the diffusion of the cells towards the underlying layers, and to form in the process a relatively smooth finished surface. Said (micro)filtration membrane may be produced based on one or more materials chosen from latex, polytetrafluoroethylene, poly(vinylidene) fluoride, polycarbonate, polystyrene, polyamide, polysulfone, polyethersulfone, cellulose and nitrocellulose.

As an alternative, WO 2015/107228, proposes covering with a porous layer of composition comprising a mixture of pigments of kaolin, talc, titanium dioxide and/or calcium carbonate and a binder of styrene-butadiene latex type, styrene acrylic latex type or carboxymethyl cellulose type.

Despite convincing biological results, such culture supports have been deemed not particularly commercially viable at the current time, especially because of a manufacturing cost that is still high.

The aim of the present invention is thus to propose a microbiological culture device which, while offering a long expiration duration, even at room temperature, provides a real alternative to conventional agar-based culture media, both in terms of fertility and compatibility with mechanical operations for streaking and spreading cells.

Another aim of the present invention is to be able to propose a microbiological culture device which is compatible with the constraints of industrial and commercial utilization, especially in terms of production cost and profitability. In particular, the present invention aims to propose a microbiological culture device with a design and manufacture that are suited to the facilities and means of production currently used in the industry of microbiological culture devices and media.

The present invention therefore proposes a microbiological culture device, comprising:
a part made of absorbent material having an at least substantially planar upper face and incorporating into its thickness a dehydrated (dry or dried) culture medium composition,
resting on said part made of absorbent material, a sheet of dehydrated polysaccharide hydrogel which can be rehydrated at room temperature, in particular at temperatures of between 5° C. and 40° C.; said sheet of dehydrated hydrogel being affixed directly on the upper face of said part made of absorbent material, or indirectly, through a permeable membrane insert.

According to the invention, said sheet of dehydrated polysaccharide hydrogel has been prepared beforehand by dehydration of a layer of hydrogel with a composition based on water and on at least one polysaccharide gelling agent. This sheet of dehydrated hydrogel has the particular feature of being rehydratable at room temperature. It regains hydrogel properties by simply absorbing an aqueous composition, without requiring any additional heat treatment, while retaining characteristics of shape, texture, hardness/stiffness and surface state that are very close to those that a sheet of polysaccharide hydrogel constituting a microbiological culture device according to the invention would have if it had not been dehydrated after pouring (or after spreading/coating) but simply hardened.

After rehydration, the sheet of dehydrated polysaccharide hydrogel once again gives a one-piece hydrogel layer, having a consistency and hardness/stiffness that are highly comparable to those of a conventional ready-to-use agar-based culture medium. In particular, said one-piece hydrogel layer has a hardness of between 500 and 2000 g.cm⁻², preferentially between 700 and 1400 g.cm⁻². The hardness of said hydrogel layer may be measured using a texture analyzer, for example of TA.XTplus type, from STABLE MICRO

### SYSTEMS LTD (United Kingdom).

During the use thereof, a microbiological culture device according to the invention must be hydrated to be activated. For this purpose, the part made of absorbent material is soaked with an amount of water or an aqueous composition. The culture medium composition, initially present in a dry state, is thus dissolved and activated. In direct contact with the part made of absorbent material, or through a permeable membrane insert, the sheet of dehydrated polysaccharide hydrogel in turn becomes rehydrated, virtually instantaneously and at room temperature. By becoming rehydrated with the liquid originating from the part made of absorbent material, the sheet of hydrogel regains flexibility and gives a thin one-piece layer of hydrogel (or hydrogel film) soaked with a dissolved and activated culture medium composition. The inoculation of the sample to be analyzed on the sheet of polysaccharide hydrogel may be carried out equally well before and after rehydration thereof. The surface of the sheet of polysaccharide hydrogel, before or after rehydration, is sufficiently smooth and stiff/hard to be suitable for operations of isolation and streaking of cells, and in particular to withstand the forces of pressure and of friction exerted by the tools and instruments developed for this purpose.

In this rehydrated system, the layer of polysaccharide hydrogel gives the surface of the microbiological culture device a lubrication effect which facilitates the mechanical operations of streaking and spreading of the cells. In addition, through its agar-like consistency, it promotes the implantation of microorganisms as close as possible to the nutrient ingredients and the active agents of the culture medium. Regarding the part made of absorbent material, aside from its role in preserving and distributing the culture medium composition, its structure contributes to the overall stiffness and firmness of the surface of the microbiological culture device, ensuring its compatibility in terms of forces of pressure and friction exerted by the tools and instruments used to streak and spread the cells.

Before continuing with the description of the invention, the definitions hereinafter are given in order to facilitate understanding of the disclosure of the invention.

The expression “culture medium” refers to a nutrient composition enabling the growth and development of cells, more particularly bacteria, molds and/or yeasts. These media make it possible to meet the nutritional requirements of the microorganisms to be cultured. In outline, the following are found in their composition:
sterile water (generally distilled or deionized water),
at least one carbohydrate, as source of carbon and energy,
and also other nutrient elements (in particular amino acids, growth factors, vitamins, minerals, trace elements, iron salts, sodium citrate, sodium chloride, etc.) provided in the form of chemically complex compositions such as mixtures of peptones (of milk, of meat and/or of potato starch, of corn, etc.), yeast extracts, serum, and/or tissue extracts of animal or plant origin, etc.,
also various salts, making it possible to establish a suitable osmolarity in the medium, and to buffer the pH.

A culture medium in the sense of the present invention may optionally exhibit a certain selectivity in terms of the target microorganisms, that is to say it promotes the growth of these target microorganisms rather than the growth of the additional flora, and/or that it inhibits and/or slows the growth of the additional flora. This selective effect may especially be obtained by virtue of the use of agents with an inhibitory effect on the additional flora or agents with an activating effect on the target microorganisms. In addition, a culture medium in the sense of the present invention may optionally exhibit differentiating abilities, making it possible to visually differentiate or distinguish between the different categories of microorganisms growing on this same culture medium. To this end, the culture medium advantageously incorporates a chromogenic and/or fluorogenic component enabling visual observation of the microorganisms as a function of the particular metabolic activities they express.

The composition and the formulation of numerous culture media are described in particular in the HANDBOOK OF MICROBIOLOGICAL MEDIA (2010; 4th Edition).

The term “sample” refers to a sample taken for purposes of analysis or to a small part or small amount of the sample taken. The invention more specifically targets biological samples containing, or suspected to contain, microorganisms to be detected and/or to be analyzed. These biological samples may be of human, animal, plant or environmental origin. They may also have an industrial origin and originate from samples taken from a manufactured product or a product in the course of manufacture or from instruments or facilities encountered in an industrial environment. The industrial sectors targeted here are more particularly the food processing, pharmaceutical, cosmetic and veterinary industries, medical devices, microbiology, and environmental testing (water, air, surfaces).

The terms “microorganisms” and “cells” are used here in an equivalent manner and refer to bacteria, yeasts, molds and/or amoebae.

“Means/tools for isolation/streaking” is intended to mean mechanical instruments able to be used for carrying out techniques of isolation (for example the exhaustion technique, streaking technique or the quadrant technique), techniques of cell coating or spreading (for example with a view to a cell count or carrying out antibiotic susceptibility tests), such as those commonly used on conventional agar-based culture media. These mechanical instruments, used manually or in an automated manner, make it possible to produce one or more point-like deposits of microorganisms on the surface of the culture medium, and by sliding over this surface they spread out the cells thereof. By way of non-exhaustive examples, mention may be made of loops, platinum loops, ground rods, beads, spreaders, pokers or rakes.

According to the invention, the proposed microbiological culture device is essentially formed of the combination between a part made of absorbent material incorporating, in its thickness, a dehydrated culture medium composition, and a sheet of polysaccharide hydrogel, also dehydrated, having the ability to be rehydrated at room temperature. By its dimensions, the sheet of dehydrated polysaccharide hydrogel covers all or part of the upper face of the part made of absorbent material.

Regarding the part made of absorbent material, which composes the inner/deep layer of the microbiological culture device according to the invention and which serves as a reservoir for a dehydrated culture medium composition, the structure, design and dimensions thereof are extensively described or suggested by EP 1 179 586, WO 2015/104501, WO 2014/013089, WO 2015/107228.

Numerous hydrophilic and non-water-soluble absorbent materials may be used to produce the part made of absorbent material of a microbiological culture device according to the invention. These materials are mainly chosen for their absorbent power, their ability to retain aqueous liquids and their ability to allow aqueous liquids to pass through them.

Advantageously and according to the invention, said part made of absorbent material is produced from a substrate of short nonwoven fibers, constituting an assembly having structural integrity and mechanical cohesion. The particularly suitable substrates are made of natural cellulose fiber (such as cotton) or synthetic cellulose fiber (such as rayon), of modified cellulose fiber (for example carboxymethyl cellulose, nitrocellulose), of absorbent chemical polymer fiber (such as polyacrylate salts, acrylate/acrylamide copolymers). According to a preferred embodiment, said part made of absorbent material is made of nonwoven textile produced from cellulose fibers, especially cotton.

A culture medium composition may be incorporated into the bulk of a part made of fibrous material in various ways.

It may be achieved by techniques of “liquid phase impregnation” (cf. for example CN 102337324 or WO 2005/061013). These techniques consist in soaking an absorbent material with a culture medium composition, formulated in solution in a volatile solvent (for example water or an alcohol). Once it has been well soaked, the absorbent material is dried by evaporation of the solvent.

It may also be carried out by techniques of “dry impregnation”. These techniques aim to transfer, into the thickness of a part made of absorbent material, a pulverulent composition, in the case in point a culture medium prepared in the form of a powder or a set of powders. To this end, the particles of said pulverulent composition are sprinkled over the surface of the part made of absorbent material, then vibrated, under the action of ultrasound waves (cf. for example FR 2 866 578) or else under the action of an alternating electric field (cf. for example, WO 2015/044605, WO 2010/001043 or WO 99/22920). These particles penetrate and then gradually sink into the cavities of the porous body.

For the production of a microbiological culture device according to the invention, the dry impregnation techniques employing an alternating electric field have proven particularly suitable to enable the incorporation of a dehydrated culture medium composition in the thickness of an absorbent material. Thus, the technical teaching provided by WO 2015/044605, WO 2010/001043 and WO 99/22920 form an integral part of the present description.

According to the invention, the amount of solution of the activated (hydrated) culture medium and the concentration of its constituents determine on the one hand the choice of the material of the part made of absorbent material (especially in terms of its capacity for water retention) and that of the dimensions of this part made of absorbent material and on the other hand the amount of culture medium powder to be incorporated therein, and vice versa.

Advantageously, before optional calendering, said part made of absorbent material has a surface density of between 50 g.m⁻² and 150 g.m⁻², and preferentially between 90 g.m⁻² and 110 g.m⁻², for a thickness also advantageously of between 0.5 mm and 10 mm and more preferentially between 1 mm and 4 mm.

According to a preferred embodiment, once the dehydrated culture medium composition has been incorporated, the part made of absorbent material is advantageously subjected to a calendering operation. Calendering, through the pressure and heating temperature generated, improves the retention of the dehydrated culture medium composition in the thickness of the part made of absorbent material, and also its stability over time. Calendering also has the advantage of improving the flatness of the surface of the part made of absorbent material, and of increasing the capillary power of same.

The calendering is advantageously carried out at a recommended temperature of between 30° C. and 60° C. A temperature of less than 60° C. makes it possible not to denature the thermolabile compounds.

EP 1 179 586, WO 2015/104501, WO 2014/013089 and WO 2015/107228, mentioned above in the present description, describe microbiological culture supports which also incorporate, in their structures, layers made of porous material incorporating a dehydrated culture medium composition. These layers thus described may therefore be taken as they are to be used in the design of the microbiological culture devices according to the present invention.

According to a particularly preferred embodiment of the present invention, the part made of absorbent material incorporating, in its thickness, a dehydrated culture medium composition advantageously has all the technical characteristics of the dry-impregnated porous support described in WO 2015/104501.

Advantageously, the amount of culture medium composition formulated as a powder and incorporated in the thickness of the part made of absorbent material is between 0.01 g.cm⁻³ and 0.1 g.cm⁻³, preferably between 0.02 g.cm⁻³ and 0.09 g.cm⁻³, and more preferentially still between 0.03 g.cm⁻³ and 0.06 g.cm⁻³.

Regarding the sheet of dehydrated polysaccharide hydrogel constituting a microbiological culture device according to the invention, the composition thereof and the thickness thereof were chosen to create a structure with a solid surface, a small thickness, and having sufficient mechanical strength and integrity to enable easy gripping and handling. Moreover, and intrinsically, this sheet of dehydrated polysaccharide hydrogel forms a hyper-absorbent material which is rehydratable at room temperature, in particular at temperatures of between 5° C. and 40° C., such that, on contact with the part made of absorbent material previously soaked with water, it becomes filled with the liquid culture medium composition and re-forms a one-piece hydrogel layer (that is to say an assembly having a certain structural integrity and mechanical cohesion) with nutrient properties, optionally selective and/or differential properties. On this hydrogel layer, the inoculated cells are thus deposited as close as possible to the constituents of the culture medium composition.

While streaking operations are often traumatic the cells, since they are generally displaced despite their adhesion to the culture support, with a microbiological culture device according to the invention, by virtue of a good surface quality of the hydrogel (optionally thixotropic properties), the cells are not brutally detached from their support but are carried along in movement with the micro-fraction of hydrogel surrounding them.

According to the invention, the sheet of dehydrated polysaccharide hydrogel is advantageously a sheet of dehydrated hydrogel of gellan, xanthan, galactomannan or starch and/or of a mixture of these hydrogels. Said sheet of dehydrated hydrogel is obtained by dehydration of a layer of hydrogel with a composition based on water and on at least one polysaccharide gelling agent. Said polysaccharide gelling agent is preferentially chosen from a gellan gum, a xanthan gum, a galactomannan gum (for example a locust bean gum or a guar gum), starch and a mixture thereof.

Advantageously and according to the invention, the sheet of dehydrated polysaccharide hydrogel is obtained by dehydration of a layer of polysaccharide hydrogel prepared beforehand by mixing 0.1 to 30 g of at least one polysaccharide gelling agent into a liter of water.

According to a first preferred embodiment, the sheet of dehydrated polysaccharide hydrogel is obtained by dehydration of a layer of gellan hydrogel, prepared beforehand by mixing 10 to 20 g of gellan gum and preferentially 13 to 15 g of gellan gum into a liter of water.

According to a variant embodiment, the sheet of dehydrated polysaccharide hydrogel is obtained by dehydration of a layer of xanthan hydrogel, prepared beforehand by mixing 0.2 to 10 g of xanthan gum, preferably of the order of 0.5 g of xanthan gum, into a liter of water.

According to a second variant embodiment, the sheet of dehydrated polysaccharide hydrogel is obtained by dehydration of a layer of xanthan and galactomannan hydrogel, prepared beforehand from a mixture of xanthan gum and locust bean gum. The [xanthan gum]/[locust bean gum] weight ratio is advantageously between 1:2 and 2:1, preferably of the order of 1:1.

According to a third variant embodiment, the sheet of dehydrated polysaccharide hydrogel is obtained by dehydration of a layer of starch hydrogel, preferably of potato starch, prepared beforehand by mixing 0.5 to 15 g of starch into a liter of water.

According to a fourth variant embodiment, the sheet of dehydrated polysaccharide hydrogel is obtained by dehydration of a layer of gellan and starch hydrogel, preferably potato starch, prepared beforehand from a mixture of gellan gum and starch. The [gellan gum]/[starch] weight ratio is advantageously between 40:1 and 2:3.

A dehydrated polysaccharide sheet can be prepared from a polysaccharide hydrogel composition in multiple ways. For example, the hydrogel may be poured or spread in a continuous layer over a non-adhering surface. This continuous layer may also be obtained by a coating method. The hydrogel layer is subsequently dried/dehydrated, then cut to the desired shape and dimensions.

The sheet of dehydrated polysaccharide hydrogel may also be prepared by a molding method, followed by step of dehydration.

According to a preferred embodiment of the invention, the sheet of dehydrated polysaccharide hydrogel is advantageously structurally reinforced chemically by means of a reinforcing additive chosen from glycerol, ethylene glycol and polyethylene glycol. Said reinforcing additive is added during the preparation of the polysaccharide hydrogel composition. Advantageously, this addition is carried out in the step of mixing the gelling agent with water, prior to the dehydration step.

In this context, a sheet of dehydrated polysaccharide hydrogel according to the invention further comprises at least one reinforcing additive chosen from glycerol, ethylene glycol and polyethylene glycol.

Advantageously according to the invention, said reinforcing additive is glycerol.

According to a particularly preferred embodiment of the invention, said sheet of dehydrated polysaccharide hydrogel is a sheet of dehydrated gellan hydrogel further comprising glycerol.

Such a sheet of dehydrated polysaccharide hydrogel is prepared from gellan gum and glycerol. The [gellan gum]/glycerol weight ratio used in this preparation is advantageously between 2:1 and 1:8, preferentially between 2:7 and 2:9, and is typically of the order of 1:4.

According to an advantageous embodiment of the invention, said sheet of dehydrated polysaccharide hydrogel further comprises at least one curing agent chosen from divalent cation salts. Preferably, the curing agent is chosen from magnesium chloride (MgCl₂), calcium chloride (CaCl₂), magnesium sulfate (MgSO₄) and manganese chloride (MnCl₂). Advantageously and according to the invention, the curing agent is MgCl₂.

Advantageously and according to the invention, said sheet of dehydrated polysaccharide hydrogel is a sheet of dehydrated gellan hydrogel further comprising at least one curing agent chosen from MgCl₂, CaCl₂, MgSO₄ and MnCl₂. The curing agent is advantageously MgCl₂. The gellan/MgCl₂ weight ratio is advantageously between 100:1 and 3:2, preferentially between 30:1 and 1:3, and is typically of the order of 15:1.

According to a particular embodiment, the sheet of dehydrated polysaccharide hydrogel is prepared from gellan gum and MgCl₂. The [gellan gum]/MgCl₂ weight ratio used in this preparation is advantageously between 100:1 and 3:2, preferentially between 30:1 and 1:3, and is typically of the order of 15:1.

According to another particular embodiment, the sheet of dehydrated polysaccharide hydrogel is prepared from gellan gum and MgSO₄. The [gellan gum]/MgSO₄ weight ratio used in this preparation is advantageously between 100:1 and 3:2, preferentially between 30:1 and 1:3, and is typically of the order of 15:1.

According to a particular embodiment of the invention, said sheet of dehydrated polysaccharide hydrogel is a sheet of dehydrated gellan hydrogel further comprising at least one plasticizer, for instance a silicone oil. The use of a plasticizer makes it possible to obtain dehydrated hydrogels with greater suppleness and flexibility. The advantage is thus being able to prepare large surface areas of dehydrated hydrogel, for example in long strips that may be placed on rolls and retained until they are cut up into sheets with dimensions suitable for the microbiological culture devices according to the invention.

The plasticizer is incorporated during the preparation of the polysaccharide hydrogel composition. Advantageously, this incorporation is carried out in the step of mixing the gelling agent and water, prior to obtaining the sheet of dehydrated polysaccharide.

According to a particular embodiment of the invention, the sheet of dehydrated polysaccharide hydrogel incorporates, in its thickness, chromogenic and/or fluorogenic compounds enabling visual observation of the microorganisms as a function of the particular metabolic activities that they express. These compounds may be, for example, synthetic substrates making it possible to demonstrate defined enzymatic activities, or colored pH indicators.

The chromogenic and/or fluorogenic compounds are incorporated during the preparation of the polysaccharide hydrogel composition. Advantageously, this incorporation is carried out in the step of mixing the gelling agent and water, prior to obtaining the sheet of dehydrated polysaccharide.

Advantageously and according to the invention, aside from the part made of absorbent material and the sheet of dehydrated polysaccharide hydrogel, a microbiological culture device according to the invention may also comprise a permeable membrane insert, arranged between said part made of absorbent material and said sheet of dehydrated polysaccharide hydrogel.

The composition, design and thickness of this permeable membrane insert are chosen such that the latter hinders the transfer of liquids between the part made of absorbent material and the sheet of dehydrated polysaccharide hydrogel or the layer of rehydrated polysaccharide hydrogel as little as possible. In this regard, it may be produced from a substrate made of natural cellulose fiber (such as cotton) or synthetic cellulose fiber (such as rayon), of modified cellulose fiber (for example carboxymethyl cellulose, nitrocellulose), of absorbent chemical polymer fiber (such as polyacrylate salts, acrylate/acrylamide copolymers) or of stable protein fibers (such as silk, wool).

In the context of the present invention, this optional permeable membrane insert may be used for highly varied purposes, for instance:
to provide additional stiffness and/or mechanical strength to the whole system, or only to the sheet of dehydrated polysaccharide hydrogel and to the layer of rehydrated polysaccharide hydrogel,
to improve the contact of the part made of absorbent material with the sheet of dehydrated polysaccharide hydrogel and/or with the layer of rehydrated polysaccharide hydrogel,
to serve as a reservoir layer for retaining particular compounds, which are intended to be mixed with the dissolved culture medium composition originating from the part made of absorbent material before being conveyed to the layer of rehydrated polysaccharide hydrogel,
to improve the contrast and observation of the colonies growing on the surface of the microbiological culture device.

According to a particular embodiment, said permeable membrane insert is used with a view to improving the contrast and observation of the colonies growing on the surface of the microbiological culture device. To this end, it is chosen to be sufficiently opaque to light and to have a high level of whiteness (for example a CIE whiteness at least equal to 65).

The design of the biological culture devices according to the invention and the modalities of use thereof make it possible for the main constituent elements, such as especially:
the part made of absorbent material,
the sheet of dehydrated polysaccharide hydrogel, and
the optional permeable membrane insert, to be able advantageously to be produced, packaged and stored separately and entirely independently. This represents a real industrial advantage, both from a commercial and logistical point of view. This also represents a significant advantage for the user, who has the possibility of combining, as desired, the different constituent elements of the microbiological culture device according to the invention and of adapting by themselves the microbiological culture device to the microorganisms in which they have a particular interest.

Similarly, a microbiological culture device according to the invention may be packaged and sold in various forms, especially:
a composite microbiological culture device, previously built and assembled, wherein the part made of absorbent material is surmounted by a sheet of dehydrated polysaccharide hydrogel, optionally with a permeable membrane insert;
a microbiological culture device in kit form, which the user will assemble themselves; such a kit comprises at least one part made of absorbent material, at least one sheet of dehydrated hydrogel, optionally at least one permeable membrane insert.

Whether the microbiological culture device according to the invention is packaged preassembled or packaged to be able to be assembled extemporaneously by a user, in order to facilitate handling thereof:
the part made of absorbent material,
the sheet of dehydrated polysaccharide hydrogel (or the layer of rehydrated polysaccharide hydrogel), and
the optional permeable membrane insert, may advantageously be held secured together by technical means advantageously applied around the perimeter of these different elements, such as:
a system of staples or pins,
an adhesive composition, applied linearly or pointwise,
a blocking system arranged inside a receptacle (for example the receptacle of a Petri dish) specifically designed to accommodate said microbiological culture device; the different constituent elements of the microbiological culture device according to the invention are, in this case, of a shape and of dimensions adapted to those of said receptacle, and the edge of said receptacle is provided on the inner face(s) thereof with mechanical holding members of lug type, protruding out of the surface.

The invention also relates to a microbiological culture device as described previously, and presented in the form of a kit to be assembled. A microbiological culture device in a kit to be assembled according to the invention thus comprises:
at least one part made of absorbent material having an at least substantially planar upper face and incorporating into its thickness a dehydrated culture medium composition,
at least one sheet of dehydrated polysaccharide hydrogel which can be rehydrated at room temperature, in particular at temperatures of between 5° C. and 40° C., and
optionally at least one permeable membrane insert.

According to a particular aspect of the present invention, said invention also proposes a sheet of dehydrated polysaccharide hydrogel which can be rehydrated at room temperature, in particular at temperatures of between 5° C. and 40° C. Said sheet of dehydrated polysaccharide hydrogel which can be rehydrated at room temperature is intended to be used as a microbiological culture support.

Advantageously, a sheet of dehydrated polysaccharide hydrogel which can be rehydrated according to the invention is characterized by all or some of the technical characteristics listed below:
said sheet of dehydrated polysaccharide hydrogel is a sheet of dehydrated hydrogel of gellan, xanthan, galactomannan or starch or of a mixture thereof,
said sheet of dehydrated polysaccharide hydrogel was prepared by dehydration of a layer of hydrogel of water-based composition and of at least one polysaccharide gelling agent chosen from a gellan gum, a xanthan gum, a galactomannan gum, starch and a mixture thereof,
said sheet of dehydrated polysaccharide hydrogel is obtained by dehydration of a layer of polysaccharide hydrogel prepared by mixing 0.1 to 30 g of at least one of the polysaccharide gelling agents mentioned above into a liter of water,
said sheet of dehydrated polysaccharide hydrogel is obtained by dehydration of a layer of gellan hydrogel, prepared beforehand by mixing 10 to 20 g of gellan gum and preferentially 13 to 15 g of gellan gum into a liter of water,
said sheet of dehydrated polysaccharide hydrogel is obtained by dehydration of a layer of xanthan hydrogel, prepared beforehand by mixing 0.2 to 10 g of xanthan gum into a liter of water,
said sheet of dehydrated polysaccharide hydrogel is obtained by dehydration of a layer of xanthan and galactomannan hydrogel, prepared beforehand from a mixture of xanthan gum and locust bean gum,
said sheet of dehydrated polysaccharide hydrogel is obtained by dehydration of a layer of starch hydrogel, prepared beforehand by mixing 0.5 to 15 g of starch into a liter of water,
said sheet of dehydrated polysaccharide hydrogel is obtained by dehydration of a layer of gellan and starch hydrogel, prepared from a mixture of gellan gum and starch, with a [gellan gum]/[starch] weight ratio of between 40:1 and 2:3,
said sheet of dehydrated polysaccharide hydrogel is structurally reinforced chemically by means of a reinforcing additive chosen from glycerol, ethylene glycol and polyethylene glycol,
said sheet of dehydrated polysaccharide hydrogel further comprises at least one curing agent chosen from divalent cation salts—especially MgCl₂, CaCl₂, MgSO₄ and MnCl₂,
said sheet of dehydrated polysaccharide hydrogel further comprises at least one plasticizer, for instance a silicone oil.

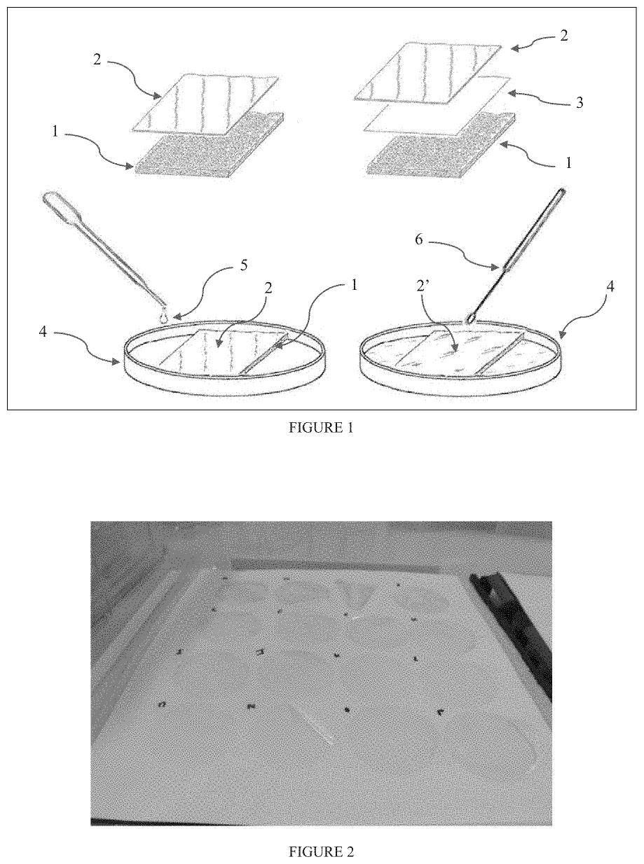

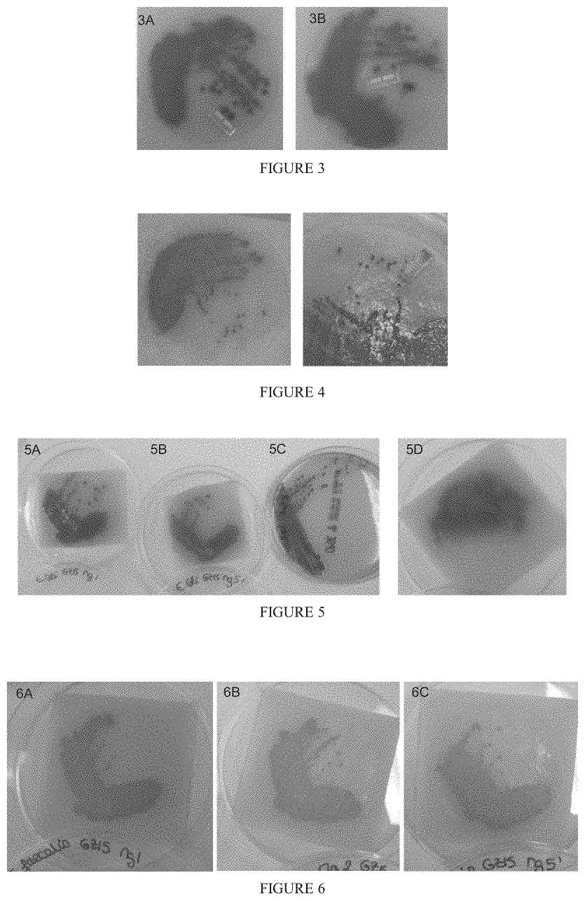

Other objectives, features and advantages of the invention will emerge in the light of the description that follows and the examples developed below, which refer to the appended figures in which:
FIG. 1 is a schematic depiction of a microbiological culture device according to the invention and the general principle of use thereof;
FIG. 2 is a photograph showing an example of sheets of dehydrated polysaccharide hydrogel at the end of the drying/dehydration process;
FIGS. 3 to 13 show photographs of cultures and isolation of bacterial strains carried out on microbiological culture devices according to the invention.

The aim of these examples is to facilitate understanding of the invention, the implementation thereof and the use thereof. These examples are given by way of explanation and cannot limit the scope of the invention.

### EXAMPLES

A/—Microbiological Culture Device According to the Invention, and General Principle of Use

As shown in FIG. 1, a microbiological culture device according to the invention is firstly composed of a part made of absorbent material 1 which is more or less thick and of a sheet of dehydrated polysaccharide hydrogel 2, of lesser thickness. In the example depicted, these two elements 1 and 2 are of substantially square shape.

The part made of absorbent material 1, made from hydrophilic and non-water-soluble material, incorporates a dehydrated culture medium composition in its thickness. The sheet of dehydrated polysaccharide hydrogel 2 is formed by drying/dehydration of a layer of polysaccharide hydrogel, and its mechanical structure can be reinforced using a fibrous reinforcement, for example a woven.

The part made of absorbent material 1 and the sheet of dehydrated polysaccharide hydrogel 2 may be provided already preassembled, secured together, or else in the form of two mechanically independent elements.

The design and the manufacture of this part made of absorbent material 1 and of this sheet of dehydrated polysaccharide hydrogel 2 will be described in greater detail in the remainder of the examples.

As secondary characteristic, a receptacle 4 is associated with the microbiological culture device in order to facilitate the handling thereof, especially for the step of rehydration and activation of the device, and for any movement (for example transfer from the lab table to the incubator).

The use of a microbiological culture device according to the invention requires activation of the device by virtue of hydration, by the part made of absorbent material 1, by water 4.

For this purpose, the microbiological culture device according to the invention is placed inside the receptacle 4, with the sheet of dehydrated polysaccharide hydrogel 2 turned upwards. Since the receptacle 4 has a larger surface area than that of the culture device, the water 5 is poured into the receptacle 4, taking care not to pour it directly on the sheet of dehydrated polysaccharide hydrogel 2. The volume of water used is more or less calibrated to be able to sufficiently moisten the part made of absorbent material 1 and dissolve the culture medium composition which it contains.

Another way of proceeding consists in pouring the suitable volume of water into the bottom of the receptacle 4, then in depositing the part made of absorbent material 1 surmounted by the sheet of dehydrated polysaccharide hydrogel 2 therein. Care will be taken not to place the sheet of dehydrated polysaccharide hydrogel 2 directly in contact with the free water, such that the device is indeed only hydrated by the part made of absorbent material 1.

When the microbiological culture device according to the invention is provided with the part made of absorbent material 1 and the sheet of dehydrated polysaccharide hydrogel 2 in the form of two mechanically independent elements, the step of hydration can then be carried out in a third way. The part made of absorbent material 1 is then placed inside the receptacle 4. In the receptacle 4 and optionally directly on this part made of absorbent material 1, a sufficient amount of water 5 is poured to thoroughly soak the part made of absorbent material 1. The surface thereof is subsequently covered with the sheet of dehydrated polysaccharide hydrogel 2.

During the activation of a microbiological culture device according to the invention, the hydration by the part made of absorbent material 1 makes it possible firstly to dissolve the culture medium composition contained in the thickness of the part made of absorbent material 1. Secondly, this activation is continued by the hydration of the sheet of dehydrated polysaccharide hydrogel 2, applied to the surface of the part made of absorbent material 1. This rehydration of the sheet of dehydrated polysaccharide hydrogel 2 causes a layer of rehydrated polysaccharide hydrogel 2′ to appear at the surface of the part made of absorbent material 1 that is swollen, not by just the water originating from the part made of absorbent material 1 but rather by a culture medium solution originating from the part made of absorbent material 1.

Thus activated, the cell culture device is ready to receive the sample to be analyzed. Once the sample has been inoculated on the device, especially by operations of streaking and spreading the cells, for example by means of a loop 6, the assembly is incubated at an established temperature and for an established duration, before reading the results.

Due to its quite particular consistency and texture, the layer/film of rehydrated polysaccharide hydrogel 2′ of a microbiological culture device according to the invention makes it possible to create a culture surface that is both lubricated and adherent for the cells, able to receive microorganisms and enable the isolation thereof by mechanical means and operations conventionally used to streak the cells over the surface of a conventional agar-based medium. Moreover, due to its large exposure to gas exchanges and to phenomena of drying out and due to its hyper-absorbent properties, this layer/film of polysaccharide hydrogel 2′ will be able to self-regenerate continuously throughout the incubation period with the culture medium solution originating from the part made of absorbent material 1.

As also depicted in FIG. 1, the microbiological culture device may also incorporate a permeable membrane insert 3 that is inserted between the part made of absorbent material 1 and the sheet of dehydrated polysaccharide hydrogel 2. Produced in a permeable material, this optional permeable membrane insert 3 may be used for highly varied purposes, for instance:
to provide additional stiffness and/or mechanical strength to the whole system, or only to the sheet of dehydrated polysaccharide hydrogel 2 and to the layer of rehydrated polysaccharide hydrogel 2′,
to improve the contact of the part made of absorbent material 1 with the sheet of dehydrated polysaccharide hydrogel 2 and/or with the layer of rehydrated polysaccharide hydrogel 2′,
to serve as a reservoir layer for retaining particular compounds, which are intended to be mixed with the dissolved culture medium composition originating from the part made of absorbent material 1 before reaching the layer of rehydrated polysaccharide hydrogel 2′,
to improve the contrast and observation of the colonies growing on the surface of the microbiological culture device.

B/—Manufacture of the Different Constituent Elements of a Microbiological Culture Device According to the Invention
B1/—The part made of absorbent material incorporating, into its thickness, a dehydrated culture medium composition

Regarding the part made of absorbent material 1 which forms part of a microbiological culture device according to the invention, said part is made from a three-dimensional support with an open, porous structure able to receive within it equally well a liquid (in particular an aqueous liquid) and solid particles with a suitable particle size.

For the examples and tests which follow, the microbiological culture devices tested comprise a part made of absorbent material incorporating a dry or dehydrated culture medium composition, produced from the nonwoven Airlaid SCA95NN81, from SCA (Sweden). This two-component PET/CoPET (Polyester/coPolyester) nonwoven was specially treated to be able to be made adhesive simply by hot pressing (calendering) without adding adhesive. It is also characterized by a surface density before calendering (or non-calendered) of the order of 95 g.m⁻² for a thickness of 2 mm. Parts with sides of approximately 6 cm were cut from this nonwoven.

The incorporation of a dehydrated culture medium composition within the very bulk of these parts made of fibrous material was carried out with a dry impregnation technique. To this end, approximately 0.2 g of a culture medium composition formulated in powder form are sprinkled over each cut nonwoven part. The assembly is placed between two electrodes applying a voltage of 3200 V.mm⁻¹, for 15 seconds with relative humidity of between 35% and 45%.

Once the dry impregnation is completed, the nonwoven parts are calendered at 60° C. by applying a pressure of 3.10⁵ Pa·cm⁻².
B2/—The culture media formulated as powder

With a view to testing the microbiological culture devices according to the invention, different dehydrated culture medium compositions were used. These have the composition of the agar-based culture media distributed by bioMérieux (France), with the exception of agar agar and other texturizing agents. These were more particularly the media of the range chromID® (for example chromID® CPS Elite, chromID® S. aureus, chromID® P. aeruginosa, chromID® VRE, chromID® Salmonella Elite).

The microbiological culture devices according to the invention, produced in this way, were then able to be tested for the detection and isolation of bacteria such as Escherichia coli, Enterococcus faecalis, Staphylococcus aureus, Pseudomonas aeruginosa, Enterobacter cloacae, Clostridium freundii, Streptococcus agalactiae and Serratia marcescens.

A selection of the results obtained is presented in FIGS. 3 to 13, in the form of photographs.
B3/—The sheets of dehydrated polysaccharide hydrogel

The sheets of dehydrated polysaccharide hydrogel 2 which form part of the microbiological culture devices according to the invention are primarily designed such that, once they are rehydrated, they can offer the microorganisms a support suited to their growth and development. The composition and the consistency of these sheets of dehydrated polysaccharide hydrogel 2 were also specifically studied to obtain surfaces suitable for operations of isolation and streaking of cells, whether these sheets of polysaccharide hydrogel are in a rehydrated or dehydrated state.

For the following examples and tests, the sheets of dehydrated polysaccharide hydrogel 2 forming microbiological culture devices according to the invention were prepared following the general method below.
a) Preparation of the polysaccharide hydrogels:
   Heat 500 ml of sterile distilled water in a glass flask on a hot plate.
   Add the gelling agent(s) in a defined amount and wait for the mixture to be homogeneous and translucent, continuing to heat (bring to boiling or close to boiling).
   After complete dissolution, add the glycerol, homogenize and add the divalent cation salt(s) serving as binder and curing agent, homogenize and bring to boiling.
   Spread 5 to 15 ml (depending on the thickness desired for the sheet) of the still hot mixture into Petri dishes 47 mm in diameter.
   Leave to cool and harden on the lab table, until the gelling agent has set.
   Remove the hydrogel parts from their mold, using a scalpel if necessary.
b) Dehydration of the polysaccharide hydrogels:
   Clamp the hydrogel parts between 2 sheets of absorbent paper.
   Place the assembly in the oven, at a temperature of the order of 40° C., for 1 to 6 hours; the operation may also be carried out for at least one night, in a dry heat oven brought to 32° C.

FIG. 2 is a photograph showing the sheets of dehydrated hydrogel on leaving the oven. By way of indication, with 7 ml of hydrogel preparation poured into the Petri dishes, the thickness of the hydrogel parts is of the order of 3 mm, before their preparation passage in the oven. After dehydration, the sheets are less than 1 mm thick.

The sheets of dehydrated polysaccharide hydrogel 2 of a microbiological culture device according to the invention were modified into different versions, by varying, especially:
the nature of the polysaccharide gelling agent (for example gellan gum, xanthan gum, galactomannan gum, starch, sodium alginate, pectin, methyl cellulose and hydroxymethyl cellulose),
the proportion between the water and the gelling agent, used during the step of preparing the polysaccharide hydrogels,
the nature and the amount of any curing agents used in the preparation of the polysaccharide hydrogels,
the nature and the amount of any additives used to improve the physicochemical properties of the (hydrated) polysaccharide hydrogels and/or of the sheets of dehydrated polysaccharide hydrogel.

B4/—The permeable membrane insert (optional)

With a view to improving contrast and the observation of the colonies growing on the surface of the microbiological culture device, a membrane that is opaque to light and with a high level of whiteness (for example a CIE whiteness at least equal to 65) may be inserted between the part made of absorbent material and the sheet of dehydrated polysaccharide hydrogel. This permeable membrane insert may consist of a sheet of absorbent paper, of cellulosic composition.

C/—Evaluation of the microbiological culture devices according to the invention

For the sheets of dehydrated polysaccharide hydrogel which have a solid surface, a structural integrity and satisfactory mechanical strength, tests were carried out with a view to evaluating their ability to form microbiological culture supports which both have good performance and are compatible with the operations and mechanical means for streaking and spreading cells.

In this context, bacterial cultures were produced especially with strains of Escherichia coli, of Enterococcus faecalis, of Proteus mirabilis of Staphylococcus aureus, of Serratia marcescens, of Pseudomonas aeruginosa, of Enterobacter cloacae, Clostridium freundii and of Cronobacter sakazaki. Depending on the bacteria of interest to be cultured, the sheets of dehydrated polysaccharide hydrogel to be tested are combined with parts made of absorbent material incorporating, in their thickness, a particular dehydrated culture medium composition. The particular feature of this dehydrated culture medium composition lies in the fact that it has been chosen to be suitable for the growth and development of the bacteria of interest, and that it incorporates chromogenic components facilitating the visual identification of these bacteria of interest.

For this purpose, the parts made of absorbent material which incorporate, in their thickness, a dehydrated culture medium composition, are placed in Petri dishes 90 mm in diameter, then moistened with 6 to 7 ml of sterile distilled water. These parts made of absorbent material are then surmounted by a sheet of dehydrated polysaccharide hydrogel. Once the hydration of the part made of absorbent material has been carried out, the culture medium has been activated and the layer of polysaccharide hydrogel has been regenerated, the microbiological culture device is inoculated by 10 μl of a calibrated solution containing a theoretical bacterial load of 10⁷ CFU/ml. The cell sample is deposited by means of a first loop on the first quadrant of the hydrogel surface. The second quadrant is inoculated with a new loop, by drawing out several streaks from the first quadrant. The third quadrant is inoculated like the second without changing the loop. The fourth quadrant is inoculated with streaks not drawn out from the second quadrant.

The dishes are placed in a jar with a small amount of water such that the microbiological culture devices do not dry out. The assembly is then incubated at 37° C. for 24 hours.

Some of the results obtained were compiled and presented in FIGS. 3 to 13 in the form of photographs.

FIG. 3 shows photographs of cultures and isolations of E. coli conducted on microbiological culture devices, the sheets of dehydrated polysaccharide hydrogel of which were obtained from 13 g/1 gellan hydrogels (that is to say 13 g of gellan gum per 1 liter of water), with a structure reinforced with glycerol, at an amount of 43 ml/l (that is to say 43 ml of glycerol per liter of water used in the preparation of hydrogel), and cured with:
3A: MgCl₂, at an amount of 1 g/l (that is to say 1 g of MgCl₂ per liter of water used in the preparation of the hydrogel) or
3B: MgSO₄, at an amount of 1 g/l.

The gellan gum used here to prepare the sheets of dehydrated polysaccharide hydrogel is Gelrite®, distributed by CARL ROTH GmbH, Germany.

Whether the curing agent is MgCl₂ or MgSO₄, the results obtained are very similar. The colonies of E. coli grow on the surface of the hydrogel sheet. They have a very good size and a very good staining. The morphotype thereof corresponds to that of colonies of E. coli growing on a reference chromogenic agar such as chromID® CPS Elite.

FIG. 4 shows photographs of a culture and an isolation of E. coli conducted on a microbiological culture device, the sheet of dehydrated polysaccharide hydrogel of which was obtained from a 15 g/l gellan hydrogel, with a structure reinforced with glycerol, at an amount of 43 ml/l, and cured with CaCl₂, at an amount of 1 g/l.

The gellan gum used here is Gelrite®.

Compared to the previous examples, the colonies of E. coli appear to be smaller, with a slight diffusion of the staining at the edge.

FIG. 5 shows a photograph of cultures and isolations of E. coli conducted on microbiological culture devices, the sheets of dehydrated polysaccharide hydrogel of which were obtained from 15 g/l gellan hydrogels, with a structure reinforced with glycerol, at an amount of 43 ml/l, and cured with:
5A: MgCl₂, at an amount of 1 g/l, or
5B : MgCl₂, at an amount of 5 g/l.

The gellan gum used here is Gelzan™, distributed by CP KELCO, United States.

With 1 g/l of MgCl₂, the colonies of E. coli growing at the surface of the hydrogel sheet have a very good size and a very good staining. The morphotype thereof corresponds to that of colonies of E. coli growing on a reference chromogenic agar such as chromID® CPS Elite (FIG. 5, part 5C).

With 5 g/l of MgCl₂, the colonies are smaller.

Without MgCl₂, the colonies are very diffuse (FIG. 5, part 5D).

FIG. 6 shows photographs of cultures and isolations of E. faecalis conducted on microbiological culture devices, the sheets of dehydrated polysaccharide hydrogel of which were obtained from 15 g/l gellan hydrogels, with a structure reinforced with glycerol, at an amount of 43 ml/l, and cured with:
6A: MgCl₂, at an amount of 1 g/l.
6B: MgCl₂, at an amount of 2 g/l, or
6C: MgCl₂, at an amount of 5 g/l.

The gellan gum used here is Gelzan™.

The colonies of E. faecalis growing at the surface of the hydrogel sheet are identical in size, color and morphotype to the colonies of E. faecalis growing on a reference chromogenic agar such as chromID® CPS Elite.

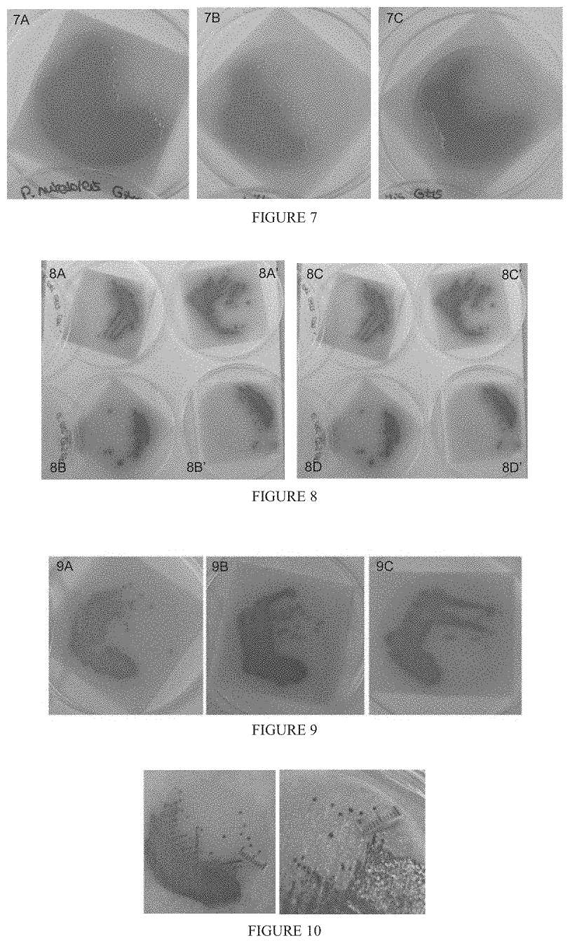

FIG. 7 shows photographs of cultures and isolations of P. mirabilis conducted on microbiological culture devices, the sheets of dehydrated polysaccharide hydrogel of which were obtained from 15 g/l gellan hydrogels, with a structure reinforced with glycerol, at an amount of 43 ml/l, and cured with:
7A: MgCl₂, at an amount of 1 g/l.
7B: MgCl₂, at an amount of 2 g/l, or
7C: MgCl₂, at an amount of 5 g/l.

The gellan gum used here is Gelrite®.

The colonies of P. mirabilis growing at the surface of the hydrogel sheet are identical in size, color and morphotype to the colonies of P. mirabilis growing on a reference chromogenic agar such as chromID® CPS Elite.

FIG. 8 shows a photograph of cultures and isolations of E. coli conducted on microbiological culture devices, the sheets of dehydrated polysaccharide hydrogel of which were obtained from 13 g/l or 15 g/l gellan hydrogels, with a structure reinforced with glycerol, at an amount of 43 ml/l and cured with CaCl₂ used at 1 g/l or 2 g/l:
8A, 8A′: 13 g/l gellan gum, 1 g/l CaCl₂,
8B, 8B′: 13 g/l gellan gum, 2 g/l CaCl₂,
8C, 8C′: 15 g/l gellan gum, 2 g/l CaCl₁,
8D, 8D′: 15 g/l gellan gum, 2 g/l CaCl₂.

The gellan gum used here is Gelzan™.

The four microbiological culture devices tested here give very similar results. Compared to the examples of FIG. 5, in which MgCl₂ is used to cure the hydrogels, E. coli here forms colonies of slightly smaller size.

FIG. 9 shows a photograph of cultures and isolations of E. faecalis conducted on microbiological culture devices, the sheets of dehydrated polysaccharide hydrogel of which were obtained from 15 g/l gellan hydrogels, with a structure reinforced with glycerol, at an amount of 43 ml/l, and cured with:
9A: 1 g/l CaCl₂
9B: 2 g/l CaCl₂
9C: 3 g/l CaCl₂.

The gellan gum used here is Gelzan™.

While the previous examples corresponding to FIG. 8 allow a prediction of a certain advantage in using MgCl₂ rather than CaCl₂ for the culture of E. coli, this observation is less obvious for the culture of E. faecalis.

FIG. 10 shows photographs of a culture and an isolation of E. coli conducted on a microbiological culture device, the sheet of dehydrated polysaccharide hydrogel of which was obtained from a 15 g/l gellan hydrogel, with a structure reinforced with glycerol, at an amount of 43 ml/l, and cured with MnCl₂, at an amount of 1 g/l.

The gellan gum used here is Gelrite®.

Compared to a culture of E coli on the reference medium chromID CPS Elite, the colonies of E. coli appear here to be smaller, but their staining is notably intensified by CaCl₂.

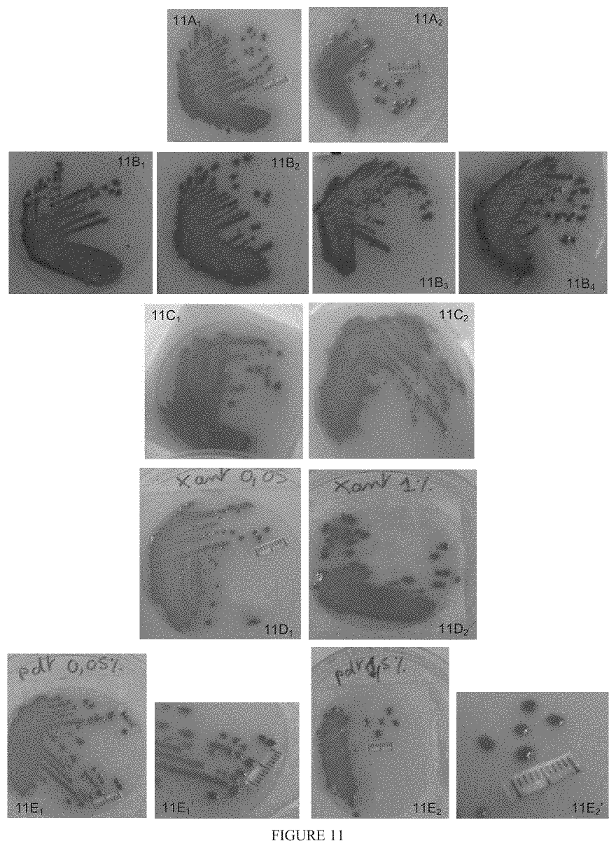

FIG. 11 shows photographs of cultures and isolations of E. coli conducted on microbiological culture devices, the sheets of dehydrated polysaccharide hydrogel of which were obtained from polysaccharide hydrogels of various compositions:
11A₁: 15 g/l gellan gum (more specifically Phytagel™, distributed by Sigma Aldrich, United States), 43 ml/l glycerol, 1 g/l MgCl₂,
11A₂: 30 g/l gellan gum (Phytagel™), 43 ml/l glycerol, 1 g/l MgCl₂,
11B₁: 25 g/l gellan gum (Gelrite®), 43 ml/l glycerol, 1 g/l MgCl₂,
11B₂: 20 g/l gellan gum (Gelrite®), 43 ml/l glycerol, 1 g/l MgCl₂,
11B₃: 8 g/l gellan gum (Gelrite®), 43 ml/l glycerol, 1 g/l MgCl₂,
11B₄: 6 g/l gellan gum (Gelrite®), 43 ml/l glycerol, 1 g/l MgCl₂,
11C₁: 20 g/l gellan gum (Gelzan™), 43 ml/l glycerol, 1 g/1 MgCl₂,
11C₂: 8 g/l gellan gum (Gelzan™), 43 ml/l glycerol, 1 g/l MgCl₂,
11D₁: 0.5 g/l xanthan gum, distributed by SIGMA ALDRICH, France,
11D₂: 10 g/l xanthan gum
11E₁, 11E₁′: 0.5 g/l potato starch, distributed by CARL ROTH GmbH, Germany.
11E₂, 11E₂′: 15 g/l potato starch.

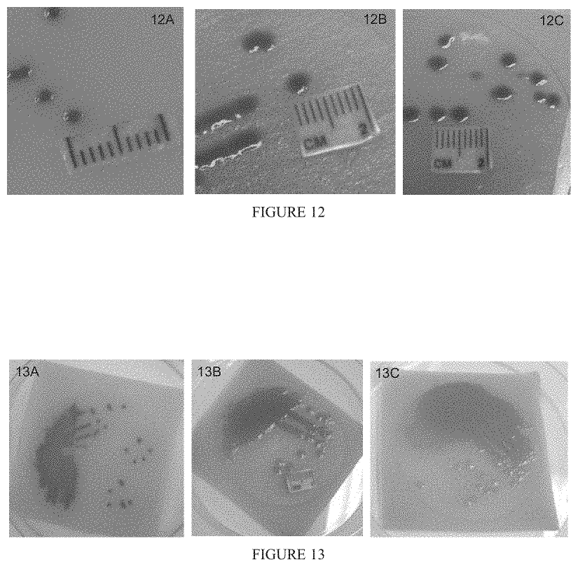

FIG. 12 shows photographs of cultures and isolations of E. coli conducted on microbiological culture devices, the sheets of dehydrated polysaccharide hydrogel of which were obtained from 15 g/l gellan hydrogels (in the case in point, Gelrite®), cured with MgCl₂ at an amount of 1 g/l and the structure of which was reinforced with glycerol at different concentrations:
12A: 32 ml/l
12B: 52 ml/l
12C: 62 ml/l

By increasing the glycerol concentration of the hydrogels, E. coli forms larger colonies, which nonetheless appear to be less protruding and more spread out on the hydrogel s.

FIG. 13 shows photographs of cultures and isolations of microorganisms conducted on microbiological culture devices, the sheets of dehydrated polysaccharide hydrogel of which were obtained from a 15 g/l Gelzan™ hydrogel, cured with MgCl₂ at an amount of 1 g/l.

These microbiological culture devices were also successfully tested for the culture and detection of Streptococcus agalactiae (13A), Serratia marcescens, (13B), and for the co-culture and co-detection of S. marcescens and S. aureus (13C).

1. A microbiological culture device, comprising:
a part made of absorbent material having an at least substantially planar upper face and incorporating into its thickness a dehydrated culture medium composition,
resting on said part made of absorbent material, a sheet of dehydrated polysaccharide hydrogel which can be rehydrated at temperatures of between 5° C. and 40° C.; said sheet of dehydrated hydrogel being affixed directly on the upper face of said part made of absorbent material, or indirectly, through a permeable membrane insert.

2. The microbiological culture device as claimed in claim 1, wherein said sheet of dehydrated polysaccharide hydrogel is a sheet of dehydrated hydrogel of gellan, xanthan, galactomannan or starch and/or of a mixture thereof.

3. The microbiological culture device as claimed in claim 1, wherein said sheet of dehydrated polysaccharide hydrogel was prepared by dehydration of a layer of hydrogel of water-based composition and of at least one polysaccharide gelling agent chosen from a gellan gum, a xanthan gum, a galactomannan gum, starch and a mixture thereof.

4. The microbiological culture device as claimed in claim 3, wherein said sheet of dehydrated polysaccharide hydrogel is obtained by dehydration of a layer of polysaccharide hydrogel prepared by mixing 0.1 to 30 g of at least one polysaccharide gelling agent into a liter of water.

5. The microbiological culture device as claimed in claim 1, wherein said sheet of dehydrated polysaccharide hydrogel is obtained by dehydration of a layer of gellan hydrogel, prepared beforehand by mixing 10 to 20 g of gellan gum.

6. The microbiological culture device as claimed in claim 1, wherein said sheet of dehydrated polysaccharide hydrogel is obtained by dehydration of a layer of xanthan hydrogel, prepared beforehand by mixing 0.2 to 10 g of xanthan gum into a liter of water.

7. The microbiological culture device as claimed in claim 1, wherein said sheet of dehydrated polysaccharide hydrogel is obtained by dehydration of a layer of xanthan and galactomannan hydrogel, prepared beforehand from a mixture of xanthan gum and locust bean gum.

8. The microbiological culture device as claimed in claim 1, wherein said sheet of dehydrated polysaccharide hydrogel is obtained by dehydration of a layer of starch hydrogel, prepared beforehand by mixing 0.5 to 15 g of starch into a liter of water.

9. The microbiological culture device as claimed in claim 1, wherein said sheet of dehydrated polysaccharide hydrogel is obtained by dehydration of a layer of gellan and starch hydrogel, prepared from a mixture of gellan gum and starch, with a [gellan gum]/[starch] weight ratio of between 40:1 and 2:3.

10. The microbiological culture device as claimed in claim 4, wherein said sheet of dehydrated polysaccharide hydrogel is structurally reinforced chemically by means of a reinforcing additive chosen from glycerol, ethylene glycol and polyethylene glycol.

11. The microbiological culture device as claimed in claim 1, wherein said sheet of dehydrated polysaccharide hydrogel further comprises at least one curing agent chosen from divalent cation salts.

12. The microbiological culture device as claimed in claim 1, wherein said sheet of dehydrated polysaccharide hydrogel further comprises at least one curing agent chosen from magnesium chloride (MgCl₂), calcium chloride (CaCl₂), magnesium sulfate (MgSO₄) and manganese chloride (MnCl₂).

13. The microbiological culture device as claimed in claim 1, wherein said sheet of dehydrated polysaccharide hydrogel further comprises at least one plasticizer.

14. The microbiological culture device as claimed in claim 1, wherein non-calendered, said part made of absorbent material has a surface density of between 50 g.m⁻² and 150 g.m⁻², for a thickness of between 0.5 mm and 10 mm.

15. A microbiological culture device in a kit to be assembled, comprising:
at least one part made of absorbent material having an at least substantially planar upper face and incorporating into its thickness a dehydrated culture medium composition,
at least one sheet of dehydrated polysaccharide hydrogel which can be rehydrated at temperatures of between 5° C. and 40° C., and
optionally at least one permeable membrane insert.

16. The microbiological culture device in a kit to be assembled, comprising:
at least one part made of absorbent material having an at least substantially planar upper face and incorporating into its thickness a dehydrated culture medium composition,
at least one sheet of dehydrated polysaccharide hydrogel which can be rehydrated at temperatures of between 5° C. and 40° C., and
optionally at least one permeable membrane insert enabling the assembly of a microbiological culture device as defined by claim 1.
